(19) United States
(12) Patent Application Publication
(10) Pub. No.: US 2025/0378150 A1
(43) Pub. Date: Dec. 11, 2025

(54) Exhaled breath based user authentication and diagnosis

(71) Applicant: Indian Institute of Technology Madras, Chennai (IN)

(72) Inventors: Mahesh V Panchagnula, Chennai (IN); Raghunathan Rengaswamy, Chennai (IN); Mukesh K, Chennai (IN)

(73) Assignee: Indian Institute of Technology Madras, Chennai (IN)

(21) Appl. No.: 19/118,363

(22) PCT Filed: Nov. 10, 2023
(86) PCT No.: PCT/IN2023/051047

§ 371 Date: Apr. 4, 2025

(30) Foreign Application Priority Data
Nov. 14, 2022 (IN) 202241065024

(51) Int. Cl. G06F 21/32; G06F 17/18; G16H 10/60; G16H 20/10; G16H 50/20; G16H 50/70; H04L 9/40

(52) U.S. Cl. CPC G06F 21/32; G06F 17/18; G16H 10/60; G16H 20/10; G16H 50/20; G16H 50/70; H04L 63/0861

(57) ABSTRACT

The present invention provides a novel user authentication system based on biometric authentication which involves user confirmation and user identification. The user authentication system is based on human exhaled breath, and executed using principles of machine learning. The user authentication system of the present invention can also be used as a diagnostic tool by the correlation of the turbulence information to the occlusion in the extrathoracic passage, which is a major source of deposition of aerosolized therapeutics. The exhaled breath time series velocity signals based diagnosis can also be used for personalized medication and treatment.

### CROSS-REFERENCE TO RELATED APPLICATIONS

The present application is a continuation of an International Application No. PCT/IN2023/051047, with a filing date of Nov. 10, 2023, the entire disclosure of which is incorporated herein by reference for all purposes. The present application claims the benefit of Indian priority application No. 202241065024, with a filing date of Nov. 14, 2022, the entire disclosure of which is incorporated herein by reference for all purposes.

### FIELD OF THE INVENTION

The present invention relates to a user identification and authentication system using a biometric system particularly related to user exhaled breath.

### BACKGROUND OF THE INVENTION

Increasing need for secured access in the current scenario has led to the development of systems that enable user identification and authentication. One such approach is the biometric authentication system. A biometric user authentication system is a real-time system that verifies a user's identity using any measured feature pertaining to the user's physiology or behaviour. Existing systems include physiological biometrics such as fingerprints, iris scans, facial recognition, etc., and behavioural biometrics such as gait analysis, voice ID, breathing gesture.

Conventional biometric systems such as voice, face, and fingerprint recognition have their own disadvantages and are susceptible to security loopholes. Established existing biometric authentication technologies, such as iris scan, fingerprint, etc., work even on dead people. Systems such as speaker recognition systems can be spoof-authenticated using recorded voices, systemized voice using deep learning techniques or even by a mimicry artist. Hence, there is a need for a better and fool-proof authentication system which can act as a real-time biometric system as well as liveness check on the subject.

Human exhaled breath is largely turbulent, as typically evident from a flow velocity signal measured using a hot-wire anemometer. During exhalation, the air is forced out of the lung through the trachea by the contracting diaphragm. As air passes through the trachea, it interacts with complex internal structures associated with the upper respiratory tract, leading to turbulent flow. The upper respiratory tract consists of the larynx, pharynx, and oral cavity, and comprises of complex morphological structures that could vary in shape and size from person-to-person.

Chauhan et al. (2017) in Proceedings of the 15^{th} Annual International Conference on Mobile Systems, Applications, and Services, Association for Computing Machinery, and Chauhan et al. (2018) in Computer, disclosed use of breathing acoustics for user authentication, wherein the biometric signature called BreathPrint® based on audio features acquired from a microphone sensor in smartphones, wearables, and other IoT devices, has been disclosed. Chauhan et al. have used a conventional machine learning model based on the Gaussian mixture model (GMM), and have established the feasibility and performance evaluation of RNN-based deep learning models.

Lu et al. in IEEE Transactions on Dependable and Secure Computing (2020), disclosed a speaker recognition system which is based on breath biometrics, wherein breath during speech which is usually considered as a trivial or a noise component is used as the signal. They have disclosed use of breath features extracted from microphone recording of speech for speaker recognition.

Respiratory flow measurements are commonly conducted using spirometers and pneumotachographs. Lafortuna et al. in Journal of Applied Physiology (1984), disclosed inspirational flow patterns in humans using measurements from a cycloergometer to theoretically estimate mechanical work. Painter and Cuningham in Respiration physiology (1992), disclosed the human respiratory flow patterns using pneumotachographic flow measurements at the mouth.

Hot wire anemometers (HWA) have been used by several researchers in the past for respiratory flow measurements. Godal et al. in Journal of Applied Physiology (1976), disclosed the application of HWA in respiratory flow measurements in small animals. However, all these studies of flow measurements were primarily focused on developing an understanding of the pulmonary system physiology.

Lundsgaard et al. in Med. Biol. Eng. Comput. (1979), disclosed the performance of a constant temperature hot-wire anemometer system (CT-HWA) for respiratory gas-flow-rate measurements. The study demonstrated that a CT-HWA meets the response requirements and is insensitive to changes in temperature and humidity that are frequently experienced in respiratory flows.

Silva et al. in Proceedings of the 19th IEEE Instrumentation and Measurement Technology Conference (IEEE Cat. No. 00CH37276) (2002), and Araujo et al. Proceedings of the 21^{st} IEEE Instrumentation and Measurement Technology Conference (IEEE Cat. No. 04CH37510) (2004), disclosed the use of CT-HWA for the measurement of fluid flow in the forced oscillations technique applied to the human respiratory system.

Kandaswamy et al. in IMTC/2002, Proceedings of the 19th IEEE Instrumentation and Measurement Technology Conference (IEEE Cat. No. 00CH37276) (2002), Xu et al. in Indoor Air (2015), and Plakk et al. in Medical and Biological Engineering and Computing (1998), disclosed the implementation of CT-HWA for measurement of expiratory flow parameters, and its potential as a flow transducer for spirography. HWA is a robust tool to obtain time-resolved turbulence signature measurements in flows.

These prior art studies have primarily used HWA data for applications such as flow rate calculations as an alternative for spirometry-based studies.

Abdelnasser et al. in Proceedings of the 16th ACM International Symposium on Mobile Ad Hoc Networking and Computing (2015), disclosed a ubiquitous WiFi-based breathing estimator ‘Ubibreathe’ that works as a non-invasive breathing rate monitoring system based on the received signal strength (RSS) data from a nearby WiFi-enabled device. The RSS at a WiFi-enabled device held on a person's chest is reportedly used to measure chest movement, from which breathing rate of the user can be inferred.

Liu et al. in IEEE INFOCOM 2020-IEEE Conference on Computer Communications, IEEE (2020), disclosed a continuous user verification system for round-the-clock user verification, built based on user-specific respiratory features that are derived from waveform morphology analysis and fuzzy wavelet transformation, wherein the breathing rate of a user, is monitored using the channel state information (CSI) of WiFi-enabled devices, again from a sensor detecting chest and abdominal motion.

Human exhaled breath has proven to be a non-invasive diagnostic tool for a spectrum of medical problems, especially when such analysis relies on the biological and chemical content of the breath.

Schaber et al. in The Journal of infectious diseases (2018), disclosed the diagnosis of malaria by analyzing the breath composition, or “breathprint”, containing volatile organic compounds produced by P. falciparum-infected erythrocytes. The researchers developed a nearest mean binary classifier with leave-1-breath-sample-out cross-validation scheme to assign predictions.

Horváth et al. in European Respiratory Journal (2017), disclosed that nitric oxide fraction in exhaled gas could serve as a potential biomarker for diagnosis of lung diseases. Mashir et al. in Advanced Powder Technology (2009), Pereira et al. in Metabolites (2015), and Das et al. in Journal of The Electrochemical Society (2020), disclosed the potential benefits of breath tests as a non-invasive technique with potential biomarkers for disease diagnosis.

Rattray et al. in Trends in Biotechnology (2014), disclosed the potential of breath-based metabolomics in personalized medicine, utilizing mass spectrometry for data profiling. Samara et al. Journal of the American College of Cardiology (2013), disclosed the enhancements required in the analysis of single exhaled breath metabolomic data for the identification of patients with acute decompensated heart failure.

These prior art studies have shown that the exhaled breath can be used as a biomarker through chemical composition analysis using various techniques, revealing compounds present in the exhaled air produce a molecular signature. However, the prior art does not provide any evidence of developing an identifier solely based on fluid dynamic aspects of exhaled airflow.

There is a strong need to develop a more sophisticated biometric system which could make use of internal physiological features of human body. Prior art studies have proposed use of techniques such as WiFi-based, and HWA for respiratory monitoring. However, none of the prior art studies have proposed the use of HWA measurements of turbulence in human exhaled breath as input signals for biometric system development.

It is hypothesized that natural inter-subject morphological variation affects the turbulent signatures in the exhaled air. A plausible way to assess this is through a user authentication system that would help classify a user purely based on the fluid dynamic signature in the exhaled breath. Two major modes of deployment of a user authentication or access system include user identification, and user confirmation. In the identification mode, a user's data is compared with registered data in the database of bona fide users, and the user is identified without the user declaring his or her identity. In the confirmation mode, a user's biometric data is compared to a specific data of the same person obtained during an enrolment process.

The present invention provides a novel user authentication system based on human exhaled breath, using the principles of multidimensional hypothesis testing and machine learning. The system is different from an acoustics-based biometric system, as it does not require the vocal data of the user and is built solely on the fluid dynamic information contained in the exhaled breath. In addition to providing biometric authentication, such a system can also find application in personalized medication, by correlating the turbulence information to occlusion in the extrathoracic passage, which is a major source of deposition of aerosolized therapeutics.

### OBJECT OF THE INVENTION

The principal object of the present invention is to develop a novel user authentication system based on human exhaled breath.

Another object of the present invention is to develop an authentication system based on the principles of multidimensional hypothesis testing and machine learning.

Yet another object of the present invention is to use exhaled breath time series velocity signals as a diagnostic tool.

Still another object of the present invention is to use exhaled breath time series velocity signals based diagnosis for personalized medication and treatment.

Still yet another object of the present invention is to use the fluid dynamics of human exhaled breath, to group or cluster humans into classes.

### SUMMARY OF THE INVENTION

The present invention provides a system that uses the human exhaled breath for authenticating a user. Said exhaled breath based user authentication system comprises receiving exhaled breath time series velocity signal by a biometric hot-wire sensor, extraction of time series velocity signals, building a library of machine learning models, and user authentication by employing embedded algorithms. Said algorithms are designed to execute different modes of authentication, namely, user confirmation and user identification. A user confirmation algorithm verifies whether said user is the person who they claim to be, and a user identification algorithm identifies a user's identity from a database with information of multiple users, without the need for the user to declare his or her identity.

The user identification algorithm aids in the establishment of two-way connectivity between users, enabling visualization of clusters among users. The clustering procedure is used as a tool to identify clusters of users from a database. Said algorithm finds application as a diagnostic tool, particularly when an individual's health baseline data is available due to probability of the turbulence information being potentially linked to occlusion in the extrathoracic passage, which is a major source of deposition of aerosolised therapeutics. The percentage of occlusion in users allows for precise medicine dosage and identification of potential diseased conditions. By creating a baseline of an individual during the healthy state, one can correlate changes to the extrathoracic morphometry in that individual through measuring changes in the exhaled breath turbulence.

### BRIEF DESCRIPTION OF THE DRAWINGS

The summary of the present invention, as well as the detailed description, are better understood when read in conjunction with the accompanying drawings that illustrate one or more possible embodiments of the present invention, of which:

FIG. 1 illustrates a calibration curve for the hot wire anemometer;

FIG. 2A illustrates the depiction of the experimental setup for data collection;

FIG. 2B illustrates a typical human exhalation velocity signal (sampled at 10 kHz for 1.5 sec);

FIGS. 3A and 3B illustrate the original and shuffled time series plots respectively representative of the effect of random shuffling of the exhaled breath time signal on the multifractal singularity spectrum;

FIG. 3C illustrates a histogram showing the distribution of all N data points of the breath signal;

FIG. 3D illustrates a multifractal spectra for the original breath signal and the randomly shuffled white noise signal;

FIGS. 4A and 4B illustrate a multifractal spectra corresponding to the entire time signal (maroon), and time segments X, Y and Z (black, bounded by gray band) respectively;

FIG. 5 illustrates a multifractal spectrum of an exhaled breath time signal with features β, ω and ϵ;

FIG. 6 illustrates a flow chart showing the algorithm pipeline towards model library building including time series normalization, filtering, feature extraction, and data splitting into training and testing;

FIG. 7 illustrates a flow chart of the user confirmation algorithm based on hypothesis testing principles;

FIG. 8 illustrates a flow chart showing the model library building procedure;

FIG. 9A illustrates a bar chart showing the percent proportion of each model in the library in the case of best-of-all model selection procedure;

FIG. 9B illustrates a box and whiskers plot showing the spread of test accuracy of each classifier;

FIG. 10 illustrates a comparison of two-dimensional decision boundaries in the (β, ω) plane, captured by different models for three randomly chosen user pairs;

FIG. 11 illustrates a user confirmation algorithm based on machine learning;

FIG. 12 illustrates a generic user identification algorithm;

FIGS. 13A and 13B illustrate the histograms of confidence of confirmation ηᵢ compared of a machine learning based approach (random forest classifiers) and a hypothesis testing based classification approach respectively;

FIG. 14 illustrates a comparison of the decision boundaries in (β, ω) plane captured by random forest classifier and hypothesis testing based classifier for a randomly chosen user pair;

FIG. 15 illustrates a plot showing the linear relation of user identification time with the growth of model library;

FIG. 16 illustrates a network chart showing various clusters of users inferred from the user identification algorithm of the present invention;

FIG. 17 illustrates a sequence of cluster charts to show the effect of threshold confidence of confirmation parameter (ηₜ) on the formation of user clusters for δ=3;

FIG. 18A illustrates a plot showing the variation of percentage of unidentifiable users with increase in the threshold parameter for different values of the minimum parity parameter (δ);

FIG. 18B illustrates a plot showing the variation of cluster connectivity strength (κ) with increase in the threshold parameter (ηₜ) for different values of the minimum parity parameter (δ); and

FIG. 18C illustrates a plot showing the variation of cluster connectivity strength (κ) with increase in the minimum parity parameter (δ) for different values of threshold parameter (ηₜ).

### DETAILED DESCRIPTION OF THE INVENTION

The present invention is related to an exhaled breath based system comprising collection of data related to exhaled breath of user, segmentation and normalization of time series velocity signals, extraction of features, building of model library to build training data for further application as a biomarker and biometric for user authentication, diagnosis, and personalized medication.

According to some embodiments of the present invention, said exhaled breath based system used for user authentication comprises confirmation and identification of a user, wherein said user authentication comprises collection of data related to exhaled breath of user, segmentation and normalization of time series velocity signals, extraction of features, building of model library to build training data, and authentication of a user based on trained data.

According to some embodiments of the present invention, said exhaled breath based system used for the diagnosis and personalizing medication, i.e., the amount of drug delivered in a user, comprises collection of data related to exhaled breath of user, segmentation and normalization of time series velocity signals, extraction of features, building of model library to build training data, identification of the user based on trained data, and classification of users based on clustering procedure.

Said exhaled breath based system for user authentication is related to a system for authenticating a user though exhaled breath of user using a hot wire anemometer (HWA) and user confirmation algorithm. According to some embodiments of the present invention, hot-wire anemometer measurements of turbulence in the exhaled breath are used as input signals for the development of a biometric system. According to another embodiment of the present invention, velocity time series can be used along with other signals such as those associated with breath related measurements that provide turbulence information in the exhaled breath. These include but are not limited to Laser Doppler Velocimetry (LDV) data, Particle Tracking Velocity (PTV) or Particle Imaging Velocimetry (PIV) data, microphone data, chemical sensing sensor data, breathing rate measurements, breathing gesture measurements, or the like.

The exhaled breath based system for diagnosis of drug delivery further comprises a clustering procedure, wherein user identification algorithm outcomes are used to identify two-way connectivity among users, enabling visualization of clusters among said users. Said clustering procedure is used as a tool to identify clusters of users from a database. Said algorithm finds application as a diagnostic tool, particularly when an individual's health baseline data is available.

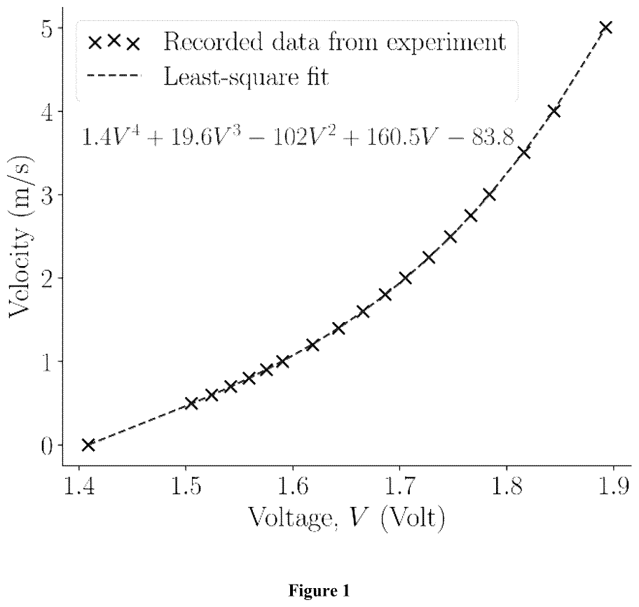

A measurement-based study is employed to develop algorithms for biometric authentication. The exhaled breath of a user is recorded using a Dantec Dynamics® 55P11 hot wire probe consisting of a 5 μm diameter, 1.25 mm long platinum-coated tungsten wire, which acts as the sensor. A Dantec Dynamics MiniCTA® 54T42 module houses a CT-HWA's signal processing and output system. Said hot wire probe is calibrated using a standard procedure of simultaneous measurement of the flow velocity and the anemometer voltage. The calibration is performed using a Dantec Dynamics StreamLine Pro® automatic calibrator, with a velocity range of 0-5 m/s. Using this procedure, the calibration constant is determined from an assumed velocity-voltage relation. This relation is a least-square polynomial fit of order-4 in the velocity-voltage space as shown in FIG. 1. The raw voltage time series is used in all the analysis which helps avoid frequent recalibration of the probe. The initial calibration is performed to ensure that the voltage and velocity signals are monotonically positively correlated (as is inferred from the least square fit shown in FIG. 1).

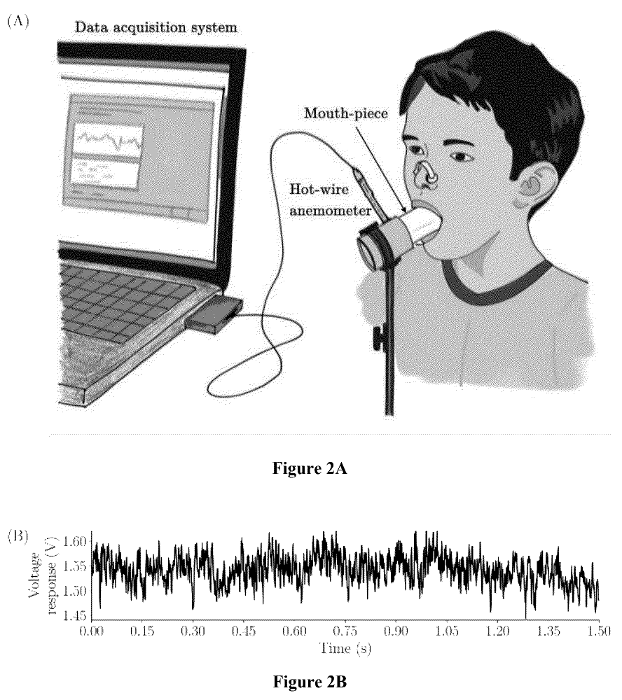

Said exhaled breath is recorded using an experimental setup as shown in FIG. 2A. It comprises a mouthpiece assembled into an aluminium circular cross-section channel housing the hot-wire probe aligned to its axis to measure the streamwise component of the turbulent exhaled flow velocity. The users exhale through their mouth into the experimental measurement setup. The nose is clipped during data recording to ensure that all the exhaled air passes through the oral cavity before entering the experimental setup. The obstruction of the tongue to the flow is avoided by placing the mouth-piece above the tongue. Data for each exhalation trial lasting about 1.5 seconds is obtained, with 10 trials recorded per subject. Each time series is recorded by sampling the voltage response at 10 kHz. This effectively gives us 15,000 data points in a single velocity time series.

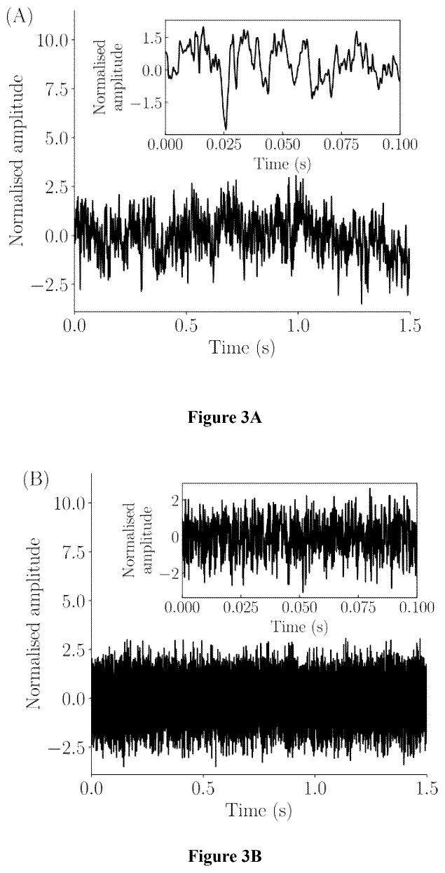

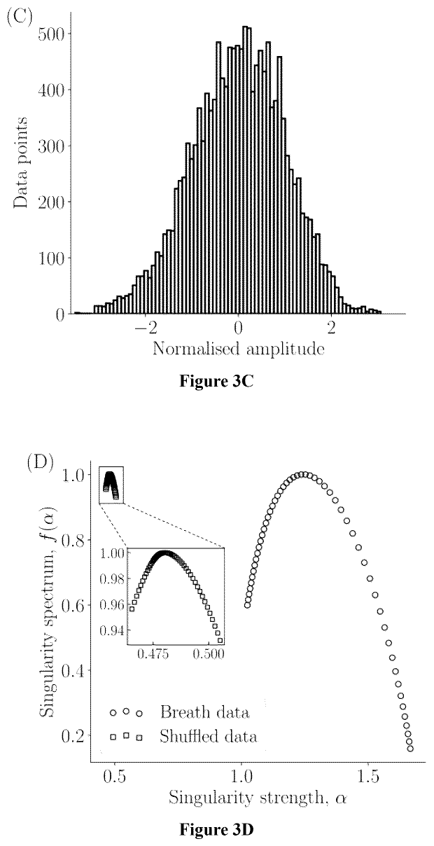

The time series velocity signal from a typical exhalation trial is shown in FIG. 3B. A library comprising the sets of time series velocity signals from multiple users is built. The user authentication algorithm comprises of segmentation, normalization, feature extraction, and subdivision of feature set into training and testing sets. Said training dataset becomes part of the enrolled database, whereas the testing dataset is used for testing the performance of the authentication algorithms. The enrolment and algorithm testing depends on the type of algorithm being used.

According to an embodiment of the present invention, the multifractal nature of exhaled breath signals is investigated using MFDFA (Multifractal Detrending Fluctuation Analysis) proposed by Kantelhardt et al. in Physica A: Statistical Mechanics and its Applications (2002). MFDFA is used to identify multifractal scaling properties as well as detect long-range correlations in a time series, wherein a Python program based algorithm developed by the inventors of the present invention is used to perform the MFDFA on exhaled breath time signals. Said algorithm divides the time series data into time intervals of equal length, applying detrended fluctuation analysis (DFA) to each time interval to remove the trend, and calculating the fluctuation function F. q-order fluctuation function F(q) is obtained by raising said detrended fluctuation function to the power of q, and q-order Hurst exponent H(q) is obtained from the scaling behavior of said F(q). Said algorithm further estimates the q-order mass exponents τ(q) from q-order Hurst exponent H(q), converting them into the q-order singularity exponents a, and computing the generalized singularity dimensions, also known as the singularity spectrum ƒ(α).

In multifractal analysis, a measure of complexity of a time series is its singularity spectrum ƒ(α), which characterizes the distribution of fractal dimensions or scaling exponents a across different parts of the signal. MFDFA provides width of the multifractal spectrum ω which indicates the richness of multifractality present in the experimental data. Third-order polynomial fits are used to detrend data in each time interval. The time interval (window) sizes range between 10 and N/4 data points, wherein N is the length of the time series. The orders q of fluctuation function ranges from −5 to 5. The input time series for the analysis is first normalized. The chosen normalization method does not alter the compact support of the input time series, as it is essential that a time series with compact support is required for reliable multifractal analysis.

FIG. 3 shows a set of plots depicting the effect of random shuffling of the exhaled breath time signal on the multifractal singularity spectrum. FIGS. 3A and 3B show the original and shuffled time series respectively. The distribution of the visualised time signal is shown in the form of a histogram in FIG. 3C. FIG. 3D is a plot of the singularity spectral function ƒ(α) against the singularity strength α, resulting from the MFDFA on the time series from FIG. 3A and FIG. 3B. The plot consists of two representative multifractal spectra—one for the exhaled breath time series and the other corresponding to the same time series shuffled, which becomes a white noise. The white noise signal is observed to form only a tiny arc clustered around α=0.5, while the multifractal breath signal forms a well-defined spectrum. The inset plot in FIG. 3D shows a magnified view of the spectrum from the white noise signal. Said white noise signal does not show any degree of multifractality, and the multifractality of exhaled velocity is defined by its inherent long-range correlation properties, both for short- and long-range fluctuations.

Segmentation of time series is crucial in data analysis and machine learning problems due to limited sample availability. Efficient segmentation based on statistical measures allows for sufficient samples for training and testing models. A 10 kHz sampling frequency provides a resolved long series for segmentation. According to an embodiment of the present invention, each time signal is divided into 19 overlapping segments using a window size of 1/10^{th} the signal length and a sliding width of half the segment size. This enables capturing the end effects of time series segments during feature extraction. The chosen segment width and sliding width are justified as each part of the time signal appears only in two segments. This results in 1500 data points per segment, allowing for 190 representative time blocks for each user analysis. The time signals are normalized before feature extraction to make the time series comparable across realizations and independent of the sensor used for measurement. The time series velocity signal can be measured using a hot-wire/film probe, or a laser-based technique. An algorithm built by the inventors of the present invention is based on features invariant to the absolute value of the time series, using z-score normalization. This involves subtracting the mean from each data point in the time series and dividing the resulting values by the standard deviation, resulting in a normalized time series with values representing the number of standard deviations away from the mean. The z-score normalization is shown in equation 1.

$$𝓏 ( i ) = \frac{x ( i ) - {μ}_{t}}{σ} , i = 1 , 2 , … N \qquad ( 1 )$$

where z(i) is the normalized time series, x(i) is the original time series of length N, (μₜ) is the mean of the time series, and (σ) is the standard deviation of the time series. The time series becomes unitless after normalization.

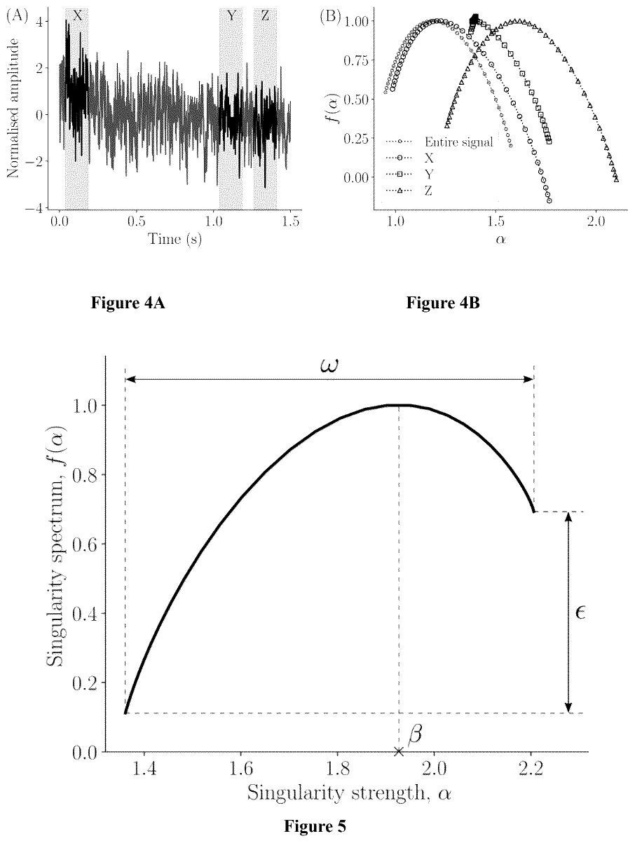

MFDFA is performed on all normalized time series and observed that not all spectra exhibit the expected shape. The general shape of a multifractal spectrum is convex or more precisely an inverted parabola, with the peak occurring at the central moment. The convex shape signifies the presence of multifractal scaling, indicating that different parts of the time series exhibit distinct scaling behaviors. Certain time segments were observed to result in a spectrum with folds or distortions. FIG. 4 shows an example of such a distortion. The multifractal spectrum for a time signal and three randomly chosen segments X, Y and Z from the same time series are displayed. FIG. 4A shows the entire time signal and the chosen segments. Out of the three segments, X and Z segments display a typical spectral shape, and segment Y displays a fold towards the left-hand side of the spectrum as shown in FIG. 4B. Folds in the multifractal spectrum are attributed to irregularities, data artifacts, non-stationarity of the time series, or the finite size of the time segment which could thereby introduce inconsistencies in scaling behavior. Said folds are indicators to judge the validity of a segment, discarding non-convex singularity spectra and segments with a spectral width less than 0.05.

According to the embodiments of the present invention, the features are extracted from normalized time signals using various time series feature extraction techniques. The input data is a time series velocity signal from a user, unlike other physiological biometric systems that use image-based patterns or features. The time series signal contains correlation structure information that is relevant to machine learning algorithms. The key features extracted from the spectrum include the abscissa corresponding to the spectral maxima (δ), the width of the spectrum (ω), and the bias or asymmetry parameter of the spectrum (ϵ), wherein said parameters are dimensionless. Said features on the multifractal spectrum of an exhaled breath time signal are shown in FIG. 5. Distinct temporal structure differences of parameters such as β, ω, and ϵ in the spectra vary for different time series velocity signals.

The “singularity strength or Hölder exponent (β)” describes the long-range correlations in the data, with lower values indicating increased regularity, potentially linked to the organization of vortical structures in turbulent exhaled airflow, which varies varying among subjects due to extrathoracic morphology.

The “multifractal spectrum width (ω)” of exhaled breath time series velocity signals characterizes the richness of multifractality in the data. A wider range of singularity strengths implies a more intricate signal structure often associated with increased turbulence in the breath flow. This heightened turbulence can be attributed to factors such as extrathoracic constriction, specific breath patterns, or dynamics, signifying diverse turbulence scales in the signal.

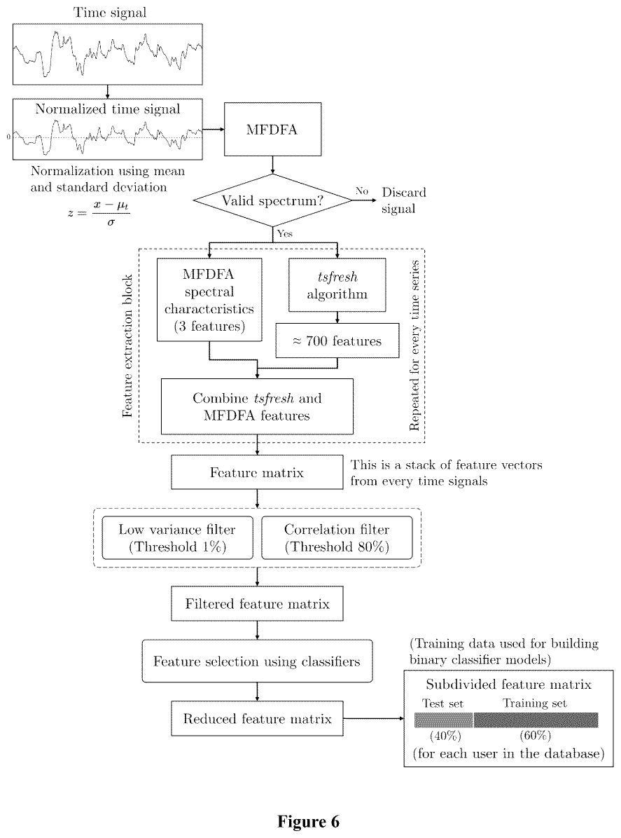

tsfresh (Time Series Feature Extraction on basis of Scalable Hypothesis tests) an automated feature extraction algorithm developed by Christ et al generates over 700 time series features using 63 different time series characterization methods. Said MFDFA and tsfresh have been used to prepare a dataset for model building, training and testing of the algorithms. FIG. 6 shows a consolidated pipeline of the algorithm for building a model library including time series normalization, filtering, feature extraction, feature reduction, and data splitting for training and testing. The time signal shown in FIG. 6 is one of the segments of the original time series; the blue bar represents the training dataset, and the green bar represents the testing dataset. The training data of all users are used for building binary classifier models encompassing the enrollment process.

Features extracted from all the available time series using the algorithms of the present invention are concatenated and passed through a low-variance filter. Feature columns with a variance value below a given threshold, which in this case was 1%, are removed. The feature set is further refined by removing highly correlated features using an 80% correlation threshold to reduce the dimensionality, simplify the model, and potentially improve model performance by focusing on the more critical features. The features derived from absolute values of the time series, such as maximum and minimum values and quantile information, are disregarded. The inclusion of a signal's mean value can bias algorithms, allowing them to classify based on the mean values, which is undesired, and hence have been excluded. Different users could exhale in varying velocity bands based on their lung capacity. The filtered feature matrix is a stack of vectors from each available time series sample consisting of around 450 time series features. The feature space is high-dimensional and may contain redundant features that can be excluded. Use of a set with reduced number of features would also decrease the computational complexity of the algorithms.

In accordance with the embodiments of the present invention, a feature selection method that uses binary random forest classifiers constructed based on pairwise combinations of user feature datasets, is adopted. The significance of features can be assessed for each random forest binary classifier by estimating the potential loss in performance if a particular feature is eliminated. The impurity-based feature importance developed by Breiman (2001) is used for determining the top features. The top 10 most common user features are selected as the feature space after listing the top 10 features from each classifier. These important time series features which have been selected automatically by the random forest classifiers, could reveal the physics behind working of the algorithm. A description of the most important classifying features is listed hereunder. It is to be noted that this list is only indicative and could be different for a different implementation as well as for a different set of users. Since this list itself is auto-generated by a Machine Learning algorithm (specifically from the Random Forest models), any of the hundreds of features describing the correlation structure in a time series are fair candidates to be included in such a list of important classifying features.

The sum of absolute consecutive velocity changes in time series quantifies the similarity between time blocks, indicating mean reversals. This metric can reveal distinct vortical structures in exhaled airflow, which are unique to each individual, and aid algorithmic classification.

The third and fourth coefficient of the autoregressive (AR(r)) model with an order parameter of 10 (AR(10)) reveals correlations between successive values in the time series, suggesting predictive patterns in the data for most of the users.

The “number of peaks” feature calculates the occurrences in a time series with a support where a value surpasses its immediate neighbors. It provides insights into localized fluctuations when said support is set to 1.

The “number of CWT peaks” feature is derived from the signal using continuous wavelet transform (CWT) with a smoothing width of 1 and 5. The Ricker wavelet evaluates the signal in both time and frequency domains. The count of distinct peaks identified across various width scales allows signal comparison based on peak characteristics, offering valuable insights into signal variability.

The “partial autocorrelation at lag 3” measures the temporal dependence in exhaled breath flow, reflecting the significance of a linear relationship between the current flow state and any step prior to the current state. It aids in understanding signal memory and is crucial for classifying human subjects based on their breath patterns.

Kurtosis of the velocity time series calculated with the adjusted Fisher-Pearson standardized moment coefficient, g2: We know that Kurtosis is a higher-order statistical attribute of velocity signals. The heaviness of the tails of the probability density functions of normalized time series could be distinct for each user. This feature will help us in assessing the degree of deviation from the Gaussian distribution and provides evidence of skewed behaviour of the time series.

The obtained reduced feature matrix contains all the desired features of all the users present in the database. The dataset is divided into training (60%) and test (40%) sets for each user, after shuffling between groups of features corresponding to the 19 time blocks for each user. The database contains 190 time signals for each user, with each set of 19 signals coming from a single recorded time series. The shuffling without grouping would lead to the same information being spread across the training and testing dataset, which is not desired. By doing this, out of 10 exhaled breath samples, 6 are included in the training set, and 4 are included in the test set. The training feature set is used to build a model library, and the test feature set is used for user confirmation and identification tests.

Binary classifier models using binary combinations of the training datasets are built to perform tests with a machine learning based algorithm, and these models are stored in a model library. Computational simulations are setup to evaluate the performance of the user confirmation and identification algorithms. The enrollment mode of the biometric system refers to the process by which the model library expands with the addition of users to the existing database. The current state of the users' database contains n disjointed users U₁, U₂, . . . , Uₙ. The complete model library can be built from ⁿC₂ binary classifier models, which can be generated from the current state of the users' database. With the addition of a user, the updated size of the users' database becomes n+1. Therefore, the size of the model library increases by n and becomes ⁿ⁺¹C₂. This growth can be expressed as shown in equation 2.

$${}^{n + 1} {C}_{2} {=}^{n} {C}_{2} + n \qquad ( 2 )$$

This shows that when a new user is added to the database, n additional binary classifier models are built and stored in the model library. Apparently, this follows a second-order power-law variation represented by y=an^{m} wherein the multiplication factor a≈0.5, and exponent m≈2.

In accordance with the embodiments of the present invention, two distinct user confirmation algorithms using the extracted feature data have been developed by the inventors of the present invention. Statistical hypothesis testing was conducted by comparing a null hypothesis against an alternative hypothesis. Development of the machine learning algorithm of the present invention comprises utilizing training data to construct random forest binary classifier models, thereby creating a library of models. Hypothesis testing-based algorithms eliminate the need for model building and the predictions are made based on hypothesis test results between a user's test data and available training data, thus making it an instance-based algorithm. Said algorithms are herein referred to as “UCA.HT” (User Confirmation Algorithm-Hypothesis Testing) and “UCA.ML” (User Confirmation Algorithm-Machine Learning). Hotelling's T² test is used in UCA.HT, which is a multidimensional version of the t-test.

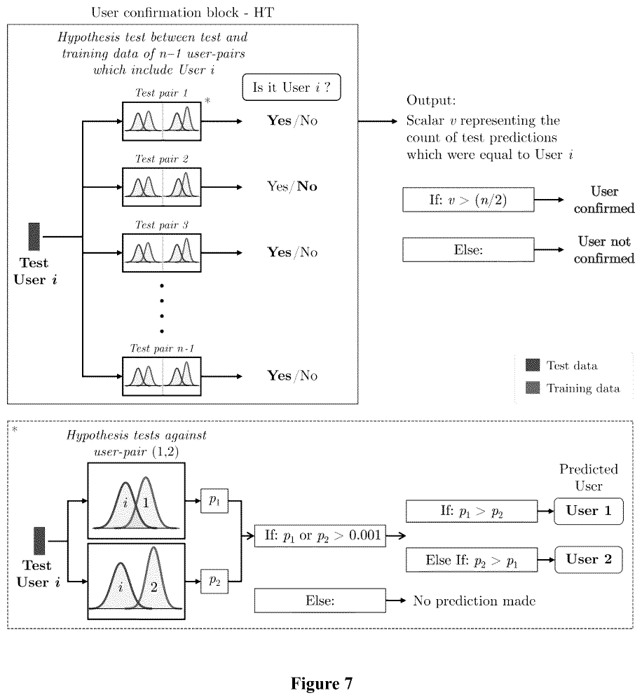

In the hypothesis testing system, the library contains training datasets of all the users. According to an embodiment of the present invention, the hypothesis testing based algorithm is formulated to work on binary pairs of users to work alongside a machine learning algorithm, wherein the library would consist of training datasets of pairs of users that would herein be referred to as user pair data. FIG. 7 shows a flow chart of the user confirmation algorithm which is based on hypothesis testing principles. The user confirmation block is used in the user identification algorithm in the present invention. An example of the hypothesis test against user pair is illustrated inside the dotted box, directed from the user confirmation block by the red asterisk. Given a user i, the user confirmation block's output is reposed to answer the question “Are you indeed User i?” based on a threshold. The equality-of-means test is performed between a test data and each training data in pairs present in the library to infer whether the null hypothesis is to be rejected or not, as depicted in FIG. 7. The null hypothesis states that the two samples come from the same distribution (H₀: μₐ=μ_{b}), and the alternate hypothesis states that the samples come from different distributions (H₁: μ₀≠μ_{b}).

Said pairwise Hotelling's T² tests compare the input of a “User i” with the training data of n−1 pairs of users, wherein n is the number of users in the database, and said training data also includes “User i”. In FIG. 7, the inside dotted box shows the hypothesis test against a user pair (1, 2) which gets a pair of p-values, (p₁, p₂). The tests are conducted with a 99.9% confidence level, indicating that a p-value of 0.001 or less is enough to reject the null hypothesis, and at least one of the two p-values must be above 0.001. The predicted user is the one with a higher p-value, and no predictions are made if both p-values are equal to or below 0.001. The test results are used to determine if the user is User i (Yes/No). The output of said “User Confirmation Block-HT” is a scalar v which is equal to the count of model predictions which says ‘Yes’. A threshold of 50% of the predictions is used for defining the minimum confidence of confirmation indicating that HT (i, i) accepts the null hypothesis, and HT (i, j) ∀j=1, 2, . . . n and i≠j rejects the null hypothesis in at least 50% of the cases, thereby confirming the User i, wherein HT (i, j) stands for hypothesis test between a User i and User j.

In accordance with the embodiments of the present invention, the equality-of-means test can be viewed from two perspectives—(a) testing the distribution of test data against n-user training data, and (b) testing the distribution of test data against training data in pairs. The former strategy produces n test results and the algorithm would face one of three scenarios: (i) If only one test accepts the null hypothesis, the user identity is assumed to be the user corresponding to that specific test; (ii) If multiple tests reject the null hypothesis, the user with the highest p-value is assumed to be the predicted user; and (iii) If all tests reject or no test rejects the null hypothesis, then the user is not confirmed. Though the former case (procedure (a)) is a computationally simpler formulation, the present invention focuses on the latter case (procedure (b)) as it aims to develop a multi-model approach for user identification. Also, the latter approach (procedure (b)) yielded superior confirmation results for UCA.HT compared to the former approach.

In accordance with the embodiments of the present invention, ⁿC₂ binary classifier models are generated to handle the multiclass problem. The choice of a classifier depends on the specific characteristics of the dataset and the multiclass problem at hand. The training dataset is used to construct binary classifier models for each user pair. The binary classifier models that may be used in accordance with the present invention include decision tree (DT), random forest (RF), support vector machine (SVM), logistic regression (LR), Gaussian naive Bayes (GNB), or multi-layer perceptron (MLP).

The performance of a user authentication algorithm is significantly influenced by the robustness of model parameters and the selection of the best model. Optimal tuning improves the generalizability of each machine learning model. FIG. 8 shows a generic algorithm of the present invention for hyperparameter tuning and model selection. The flow chart shown in FIG. 8 represents the model library building procedure for a user pair (i, j), wherein i=1, 2, . . . n; j=1, 2, . . . n, and n is the total number of users. The model mᵢⱼ=mⱼᵢ indicates that only model mᵢⱼ is created and stored in the library. The training data for a user pair (i, j) is normalised initially using the mean (μᵢⱼ) and standard deviation (σᵢⱼ) of the training set. μᵢⱼ and σᵢⱼ is stored in the memory for scaling the test data. It can be combined into a function called standard scaling function s(μᵢⱼ, σᵢⱼ) for later use. The normalized training data is utilized for tuning and training the best model. It is challenging to know the values of the model parameters for a given machine learning model on a dataset. So, the present invention utilizes an iterative search cross-validation scheme to compare various hyperparameter values for each model. A stratified k-fold cross-validation technique with hyperparameter tuning is employed for evaluation and selection of the model parameters. The number k specifies the number of times the algorithm has to split the training dataset to validate and generalize the model. The number (k) of folds is chosen to be 5 in the present invention as small k may not generalize well and larger k would lead to longer computation. Any k value in the range between 2 and 10 is typically acceptable. Parameters from a hyperparameter search space are fed into the cross-validation algorithm, wherein the training data is split into k equally sized folds maintaining the same target class distribution in each fold as the original dataset and ensures that there is no class imbalance in each of the k folds. Bayesian search is employed when the search space is large, whereas the grid search is employed for smaller search space where the method of brute force is computationally affordable. During an iteration, for a selected hyperparameter configuration, the model is trained and evaluated k times, each time using a different fold as the validation set and the remaining k−1 folds as the training set. The set of parameters with best cross-validation score are selected, and the model is retrained on the normalized training data based on the selected parameters. The standard scaling function and the classifier model are combined into a model pipeline {s(μᵢⱼ, σᵢⱼ), mᵢⱼ}, wherein mᵢⱼ is the classifier built corresponding to user pair (i, j). The pipeline is designed to ensure that test data is scaled using the mean and standard deviation of the training set before making predictions.

Said procedure for the binary classifiers, effectively reduces the feature set size from approximately 450 dimensions to 10 dimensions using random forest classifiers. The number of trees/estimators are tuned. The splitting rule is tuned by controlling the maximum depth of a tree, minimum number of samples required to split an internal node, and minimum number of samples required to be at a leaf node. The selection of the optimal model based on performance is crucial for creating an efficient library of best estimators for the training data. The ⁿC₂ models are subjected to hyperparameter tuning before fitting to the training data to ensure that they are generalized for the corresponding user pair's data. The model with a cross-validation score of 60% or less is discarded to ensure that the models saved in the library are not random. This leads to the overall algorithm performing reasonably well to good by storing models with a cross-validation score above 60%.

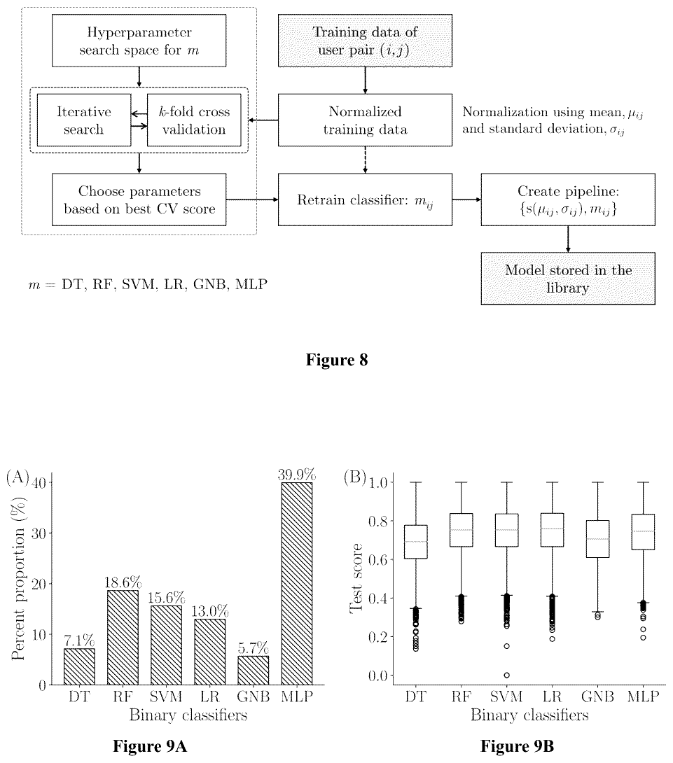

In accordance with the embodiments of the present invention, the best-of-all model selection technique involves selecting the binary classifier model with the highest cross-validation score for a specific user pair. FIG. 9A shows the percentage proportion of various models in a library constructed using this procedure. The MLPs are the most frequently used best classifier, followed by RF, SVM, LR, DT, and GNB, based on their highest cross-validation score. The process of building all the six models and choosing the best one every time is computationally very expensive. So, one out of the six classifiers is to be chosen for testing the algorithms. The present invention relates to a system comprising a procedure that alleviates this problem. The information presented in FIG. 9A is insufficient for decision-making, as a good cross-validation score doesn't guarantee good performance on test data, and classifiers may overfit the training dataset. Pairwise user test data is used to test each user classifier, and the results are shown as a box and whiskers plot in FIG. 9B. The orange line inside the boxes represents the median of the test score. It is clear that DT and GNB classifiers perform poorer than the others, while RF, SVM, LR, and MLP have very similar performance on the test data. All the models have produced test accuracies ranging from very low values below 0.5 to 1, which is excellent. The outlier data points reveal that SVM does very poorly as the accuracy even goes below 0.2, and LR and MLP too have produced accuracies below 0.2. The RF classifiers and GNB have similar lower bound of test accuracy.

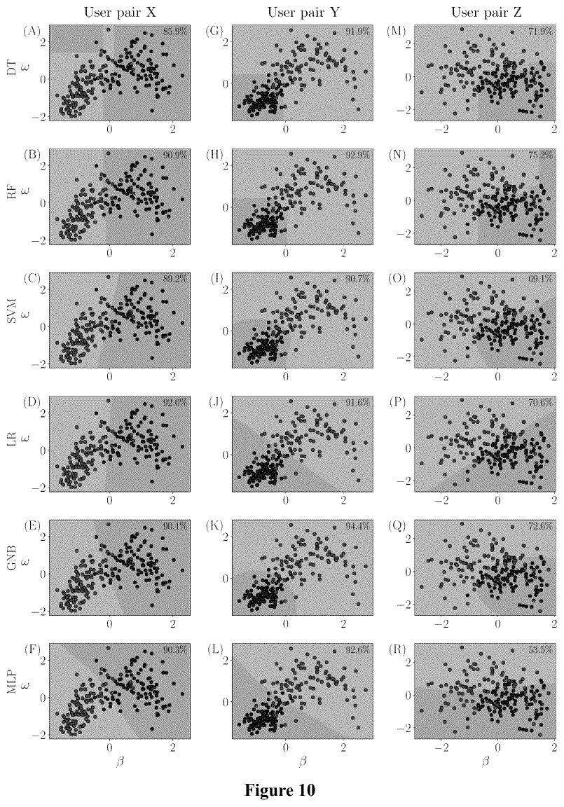

Visualizing the decision boundaries in a 2D feature space helps understand how these models fit training data. In accordance with the embodiments of the present invention, any 2 dimensions out of it for building models and visualization are chosen so as to reduce the feature space of 10 dimensions to become visualizable. The (β, ω) space is chosen for visualisation. To generate the 2D decision boundaries, a structured synthetic dataset is generated which fills up the two-dimensional feature space within the given bounds. The decision regions are obtained based on the predictions made on each data point from the synthetic dataset. These boundaries for three randomly chosen user pairs are shown in FIGS. 10A to 10R for comparison.

FIGS. 10A to 10R show two-dimensional decision boundaries. The scattered points are the training data points with red and blue labels denoting their true classes respectively. The line separating the two contour regions is the decision boundary. A region R in the feature space is classified as a decision region under class yᵢ(i={0, 1}) if all the samples xⱼ in that region is classified as yᵢ. A decision boundary separates these 2 decision regions. Therefore, the feature space is divided into two parts by the decision boundary for a binary classification problem. This representation aids in visualizing class distinctions and comparing multiple binary classifiers and their decision mechanisms. The scattered points in each plot of FIG. 10 represent the training data points with their respective colors corresponding to two users. The test data accuracy for each model is displayed at the top right corner of the corresponding plots. The user pair X (shown in FIGS. 10A to 10F) shows that all the models perform well, with LR and RF scoring higher than other choices. Similarly, for user pair Y (shown in FIGS. 10G to 10L), all the models perform well, with GNB, MLP and RF producing the best scores. The decision boundaries captured by SVM (shown in FIG. 10H) and RF (shown in FIG. 10K) appear similar, with small variations in the captured boundaries leading to one algorithm performing better than the others. The models show lower accuracy against test data for user pair Z (shown in FIGS. 10M to 10R) compared to user pairs X and Y. GNB and RF produce the highest scores among the models. It is shown in FIGS. 10B, 10H, and 10N that the random forest models demonstrate superior performance in capturing complex decision boundaries in all the three cases, possibly due to their robustness to outliers through bootstrapping and ensemble schemes. Random Forest (RF) is chosen as the optimal binary classifier model for the model library due to its ability to reduce overfitting risk by aggregating predictions from multiple decision trees and generalizing well. All the ⁿC₂ trained models are stored in the library, wherein n is the total number of users. The random forest is used as the machine learning algorithm for user confirmation and user identification.

In accordance with the embodiments of the present invention, after building the model and storing the entire library, a particular user data, specifically “User i”, is input, and the algorithm selects those models from the library which are built using the same test user and makes predictions using each model as depicted in the flow chart in FIG. 11. The predictions provided are responses to the question “Is it User i?” (Yes/No). The pipeline discussed has been renamed as ‘User confirmation block—ML’. The output of this block is a scalar v which is equal to the count of model predictions which says ‘Yes’. A threshold of 50% of the predictions is used for defining the minimum confidence of confirmation. This means that if the algorithm confirms the user in more than half the classification trials, i.e., when v>(n/2), the user is confirmed, else not.

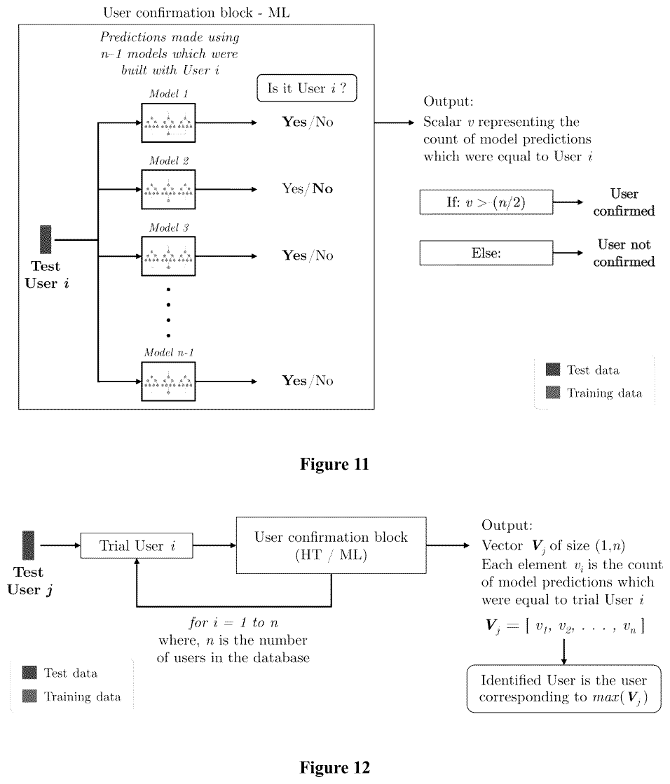

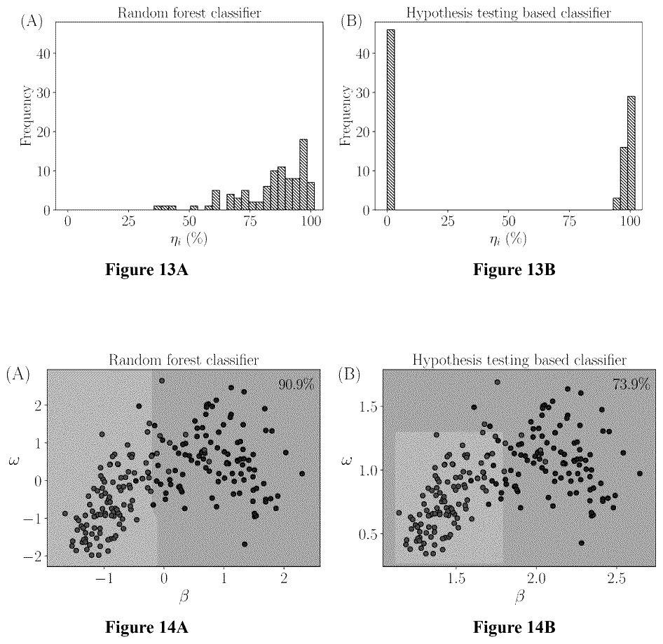

The present invention is related to a novel biometric system that relies solely on human exhaled breath for user identification without the need for the users to disclose their identity. A major challenge with a user identification system is to test its performance. The confirmation algorithm aims to verify if the user is “User i”, while the identification algorithm addresses the more general authentication question “Who is the User?”. The machine learning algorithm utilizes said model library for making predictions. FIG. 12 shows a generic user identification algorithm. The user identification algorithm includes a user confirmation block (either HT from FIG. 7, or ML from FIG. 11) during the identification of a specific user. This is equal to effectively running through all the ⁿC₂ models present in the library, but in batches of trial users, User i, wherein i=1, 2, 3, . . . n. The output of this pipeline is a vector V of size (1, n) with each element vᵢ being a result of the corresponding trial confirmation test. The identified user from this algorithm would be the trial user corresponding to the maximum value in the vector V. When more than one confirmation trial results in the maximum prediction value (two elements of V having the maximum value), the algorithm does not identify any user. The user identification algorithm is generic, which means that any user confirmation algorithm (instance-based or model-based) can be used within this algorithm and the output of this algorithm would be vector V containing the count of predictions. This enables the creation of a multi-model approach for user identification, enabling the combination of multiple identification results through a weighted sum. This encompasses combining the results from multiple expert units. The outputs from hypothesis testing based and machine learning-based user identification algorithms, are herein referred to as V^{HT} and V^{ML} respectively. The weighted sum of the two vectors can result in a new vector V′, incorporating the benefits of both the algorithms, as illustrated in equation 3:

$${V}^{′} = {w}_{1} \times {V}^{HT} + {w}_{2} \times {V}^{ML} \qquad ( 3 )$$

wherein, w₁ and w₂ are the weights associated with hypothesis testing based algorithm and the machine learning based algorithm, respectively. The values of the weights can be between 0≤w≤1, and sum of the weights should always sum up to 1. The approach can be extended to a combination of multiple user identification algorithms, as illustrated in Equation 4, wherein there are r output vectors V₁, V₂, V₃, . . . , Vᵣ from r algorithms,

$${V}^{′} = {w}_{1} \times {V}_{1} + {w}_{2} \times {V}_{2} + {w}_{3} \times {V}_{3} + … + {w}_{r} \times {V}_{r} \qquad ( 4 )$$

In accordance with the embodiments of the present invention, confirmation tests are performed for all users (n) available in the database. Each set of confirmation tests is repeated sufficient number of times by shuffling training and test data split-up. The results of the algorithm from each of these trials can be interpreted as number of confirmed users denoted by c, and number of unconfirmed users denoted by u. In order to quantify the performance of the algorithms, it is defined aa a metric called the true confirmation rate (TCR) which is a ratio of the confirmed users and total number of users as shown in Equation 5.

$$T C R = \frac{c}{n} \times 100 \qquad ( 5 )$$

The confidence of confirmation (n) for a user confirmation algorithm is the percentage prediction of the favourable user during a confirmation test. It directly quantifies how confident the algorithm is while attempting to confirm a user i. It is defined as

$${η}_{i} = \frac{v}{n - 1} \times 100 \qquad ( 5 )$$

wherein, vᵢ is the favourable user predictions as can be seen in FIGS. 7 and 11, i.e., the total number of model predictions that matches the user that the algorithm is attempting to confirm, and n is the total number of users in the database. The value of ηᵢ ∀i=1, 2, . . . n must pass a threshold confidence of confirmation, for example ηₜ, for a user to be confirmed.

FIG. 8 shows comparison of histogram of the confidence of confirmation ηᵢ. Histograms of confidence of confirmation ηᵢ compared between a machine learning based approach (random forest classifiers) (A), and a hypothesis testing based classification approach (B), for one trial of n confirmation tests. The predictions from ML classifiers produce a range of ηᵢ values distributed between ≈38% to 100%, whereas the predictions from HT based classifiers produce ηᵢ values close to 0% and 100%. It is observed that machine learning-based algorithms outperform hypothesis testing-based algorithms. The random forest classifier captures decision boundaries more effectively when compared to its hypothesis-testing-based counterpart. For the UCA.HT, the TCR is 50±10%, whereas, for the UCA.ML, the TCR is 97±2.5%. This implies that almost every user can pass the threshold of 50% in the machine learning based algorithm that signifies the algorithm. This creates a greater level of confidence while confirming a user using UCS.ML.

In hypothesis testing, the null hypothesis is rejected based on the confidence level chosen that can be shown as a demarcating hyper-surface between two n-dimensional normal distributions. FIG. 14 shows the comparison between random forest classifier (A) and hypothesis testing based classifier (B) in a two-dimensional feature space to examine their decision boundaries in (β, ω) plane; and decision boundaries that are captured by random forest classifier and hypothesis testing based classifier for a randomly chosen user pair. The scattered points are the training data points with red and blue labels denoting their true classes respectively. The class regions are computed using a structured synthetic dataset in the feature space. The line separating the two contour regions is the decision boundary. Accuracy of each model against the test data is shown at the top right corner of their respective plots. The RF classifier captures a complex decision boundary compared to the HT based classifier. The decision boundary of a hypothesis testing based classifier is visualized by performing z tests in each dimension separately, for every data point from the synthetic dataset against one of the user's training data. The tests are performed under the null hypothesis that the data point belongs to the distribution of the training data, under a confidence level of 99.9%. The overall null hypothesis is accepted only if the null hypothesis in both the dimensions are accepted. The random forest model effectively captures complex decision boundaries for the same pair of users compared to the hypothesis testing based algorithm. The random forest classifier achieves a test data accuracy of 91%, outperforming the hypothesis testing-based classifier which achieves only 74%. Therefore, the machine learning-based algorithm outperforms the hypothesis testing based algorithm for user confirmation.

The results of the user identification system in accordance with the various embodiments of the present invention, are presented here. The identification algorithm shown in FIG. 12 depicts obtaining a vector V of favourable user predictions. Based on the attributes of Vⱼ, the following outcomes are obtained:
True positives (t)—Number of users identified correctly;
False positives (f)—Number of users identified incorrectly; and
Not identified (h)—Number of users not identified by the algorithm.

The performance metrics considered for the evaluation of the user identification algorithm include Precision (P) or Positive Predictive Value (PPV), and Accuracy (E). Precision (P) or Positive Predictive Value (PPV) represented by equation (7) quantifies the percentage of users identified correctly among all the identified users.

$$P = \frac{t}{t + t} \times 100 \qquad ( 7 )$$

This parameter measures the likelihood of the algorithm correctly predicting a given judgment or identification.

Accuracy (E) represented by equation (8) quantifies the percentage of users identified correctly from the database of users

$$E = \frac{t}{n} \times 100 \qquad ( 8 )$$

Using the hypothesis testing based algorithm represented by Equations (7) and (8), a precision of 35±11%, and an accuracy of 29±9% was achieved. The results are presented in the format ‘μₚ±2σₚ’, wherein μₚ and σₚ represent the mean and standard deviation of the performance metrics respectively. Using the random forest-based algorithm, a precision of 26±7%, and an accuracy of 22±6%, was achieved. the maximum votes received by a user among n confirmation trials is computed using the algorithm shown in FIG. 12. The combined results using Hypothesis testing based algorithm and Random forest-based algorithm and considering w₁=0.3 and w₂=0.7 of equation (3) produced a precision of 32±8.5%, and an accuracy of 31±8.5%. Said values are also influenced by the threshold ηₜ, which is set to 55% in this case. The parameters w₁, w₂, and ηₜ make the algorithm behave on both extremes, i.e., either very liberal (low precision, low accuracy), or very conservative (high precision, low accuracy). In an example accordance with the embodiments of the present invention, wherein n=94, weight w₁=0.3, weight w₂=0.7, and ηₜ=50% produces the outcomes (t, f, h)=(31, 58, 5), with a precision of 34.8% and accuracy of 33.0%. In another example in accordance with the embodiments of the present invention, wherein n=94, weight w₁=0.3, weight w₂=0.7, and ηₜ=96% produces the outcomes (t, f, h)=(18, 6, 70), with a precision of 75.0% and accuracy of 19.1%. The judgements while using Hypothesis testing based algorithm often led to false positives, whereas the judgments using Random forest-based algorithm are more stringent. It must be noted that the values of the hyperparameters indicated above are representative and can vary from implementation to implementation without loss of generality of the said invention.

According to an embodiment of the present invention, a multi-model approach, with the appropriate hyperparameters w₁, w₂, . . . , wᵣ (in the general case, from equation 4) and ηₜ, is anticipated to enhance the overall robustness of the algorithm. If one classifier produces incorrect predictions for certain trials, other classifiers in the ensemble can compensate for it and provide correct predictions. The contribution of each algorithm can be controlled by the weights. This robustness helps in improving the generalization of the ensemble model.

In accordance with the various embodiments of the present invention, the produced combined results of hypothesis testing based algorithm and random forest-based algorithm are presented here. The highest voted user becomes the identified user from the algorithm. Sufficient number of shuffle trials were conducted, which projected 21% to 43% as highest voted users, i.e., 21% to 43% of the users were correctly identified, 39% to 57% of the users were correctly identified at least as the second highest voted users, and 50% to 66% of the users were correctly identified at least as the third highest voted users.

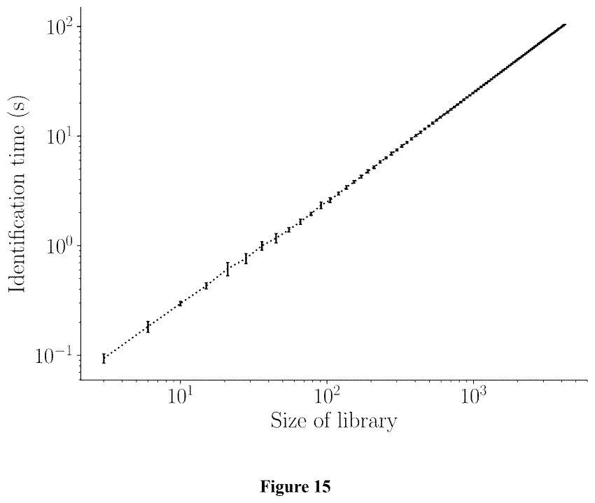

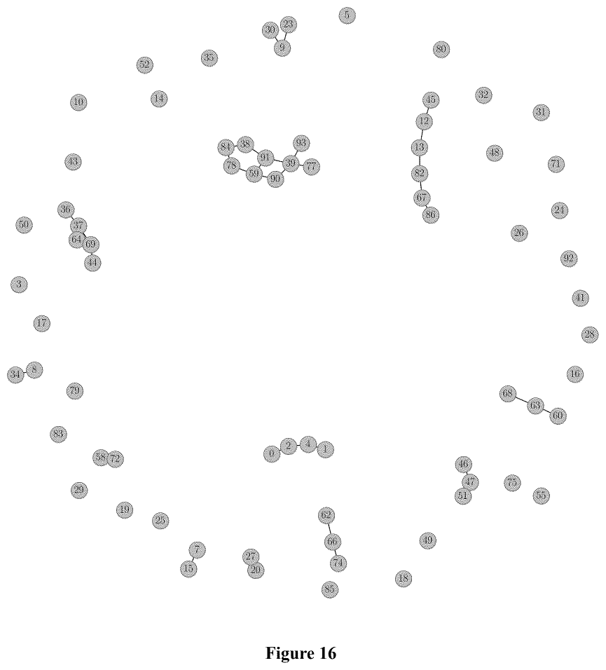

The run-time of an algorithm is a crucial element in a real-time biometric system. The size of the input feature set affects the extent of computational resources required to run an algorithm. It is observed that the hypothesis test-based algorithm performs predictions faster than the machine learning based algorithm as it is an instance-based classifier. The efficiency of the user identification algorithm is expected to increase with the size of the library, as it depends on the number of users and models available in the model library. The identification time is found to be linearly related to the library size, as depicted in FIG. 15. This pertains to machine learning-based algorithms that involve creating binary classifier models, also known as enrollment in biometrics context, and the error bars display a 95% confidence interval at every data point. An algorithm using ⁿC₂ binary classifiers instead of a single multi-class classifier is massively parallelizable, and improves the computational time by several orders if sufficient cores are available for model loading and prediction. It significantly improves the computational time by several orders and can also be implemented in a cloud computing infrastructure.

It is believed that the present invention is first of its kind attempt to classify and uniquely identify individuals based solely on the fluid physics of exhaled breath. The fluid dynamic structure of exhaled breath is believed to contain unique, identifiable information. The algorithm has significant potential for future use in personalized medicine and as a novel method for storing biological data. This can be achieved by careful model selection and generalization of classifier models. Advanced models such as deep neural networks can be used to enhance the multi-model approach.

According to another embodiment of the present invention, the uniqueness of human exhaled breath velocity signals can be used as a biomarker to store biological data of humans. The clustering analysis results could aid in categorizing humans based on exhaled breath signals. By grouping or identifying breathing classes with similar dynamics, the breathers can be labeled, to personalize the medication prescribed to them. The present invention has potential for application in future diagnostic systems wherein a patient walking in, exhales into a device as described in the present invention, which can be analyzed by a clinician considering the user's exhaled breath characteristics and utilizing the learnings from standard human user clusters to refine and deliver customized medication.

According to another embodiment of the present invention, the exhaled breath time series velocity signals can also be used as a diagnostic tool, and aid in personalizing medication. A user identification algorithm as depicted in FIG. 12 is used for the purpose. Said user identification algorithm incorporates a user confirmation block during the identification of a given user. When a new test user data is provided as input, for example, ‘User j’, the algorithm runs the user confirmation block by considering all the users in the database as trial users. The output of this block for each trial is a scalar v which is equal to the count of acceptable model predictions. Thus, this procedure is as effective as running through all the ⁿC₂ models present in the library, but it is conducted in batches of trial users, User i, wherein i=1, 2, 3, . . . , n. The output of said process is a vector V of size (1, n) with each element vᵢ being a result of the corresponding trial confirmation test. All such vectors Vⱼ, wherein j=1, 2, 3, . . . , and n are stacked as rows in a matrix. This matrix may be considered as the user identification matrix (matrix A). Matrix A is used to understand the relationship between users. The user clusters are found using the information from said matrix using the clustering procedure in accordance with some embodiments of the present invention.

Said clustering procedure comprises calculation of user identification matrix A, parity matrix P, and confusion matrix C.

Said user identification matrix A is represented by

$$A = {[ \begin{matrix}… & {V}_{1} & … \\ … & {V}_{2} & … \\ … & {V}_{3 \vert} & … \\ & ⋮ & … \\ … & {V}_{n} & …\end{matrix} ]}_{n \times n}$$

wherein, the rows represent the true users, and columns represent the trial users. The size of the matrix is n x n as every user from the database is tested against every other user.

### Parity Matrix P is Represented by

$$P = A - {A}^{T}$$

However, Parity matrix shows results of a trial user i is identified v times when testing for a true user “user j”, and vice versa. If P(i, j) is zero or close to zero, it indicates similar working of user confirmations between a specific user pair, and a threshold can also be set for these values to have a control on the extent of dissimilarity between a pair of users.

Confusion matrix C establishes a two-way connectivity between two users, and is represented by

$$C ( i , j ) = 1 , if it meets ( A ( i , j ) > {η}_{t} ) and ( A ( j , i ) > {η}_{t} ) and \vert P ( i , j ) \vert \leq δ , else C ( i , j ) = 0 ,$$

wherein i=1, 2, . . . , n, and j=1, 2, . . . , n; ηₜ is the threshold confidence of confirmation; and § is the minimum parity parameter which is an integer.

Said confusion matrix therefore consists of only 0s and 1s, wherein the 1s represent similarity between the user pairs. The parameters ηₜ and δ are tuned to understand the clustering of users.

FIG. 16 illustrates a network chart showing various clusters of users inferred from a user identification algorithm. The clusters of users are identified based on the clustering procedure of the present invention. Said network chart is a visualization of the clusters using nodes and linkages. The python package NetworkX developed by Hagberg et al. (2008) is used for visualizing these clusters. Each node in this network chart is a user, and the linkage drawn between them shows a two-way connectivity between two users. The cluster diagram of FIG. 16 shows the connectivity between the users through a linkage and are indistinguishable by the algorithm as they share similar characteristics in their exhaled breath. FIG. 16 also shows that not every user in a cluster has two-way connectivity with every other user in the cluster. For example, from the 9-users cluster presented on the chart, it is observed that user pairs ‘39-93’ and ‘39-77’ share similar characteristics and have established a two-way connectivity, but user pair ‘93-77’ does not share any similarity. There are few more clusters which are of different sizes. It can also be observed that a few users do not fall into any of the clusters. These users are clearly distinguishable from every other user from the database. This provides visual evidence on humans falling into different categories in terms of breathing as well as extrathoracic morphometry.

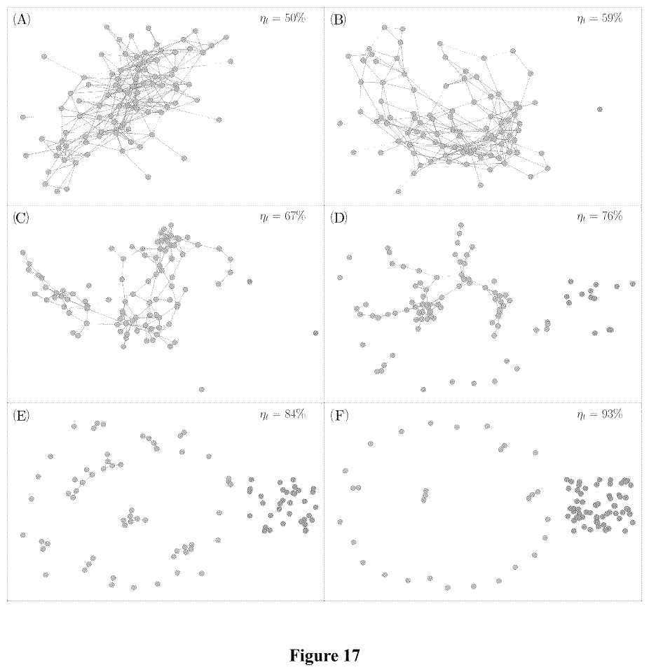

According to an embodiment of the present invention, a quantitative cluster analysis is conducted considering two parameters ηₜ and δ that denote the threshold confidence of confirmation (in %) and minimum parity, respectively. FIGS. 17A-17F illustrate a sequence of cluster charts to visualize the effect of threshold confidence of confirmation parameter (ηₜ) on the formation of user clusters for δ=3, wherein each node represents a user, and sky-blue and red nodes represent identifiable users and unidentifiable users, respectively. The number of clusters increase with increase in ηₜ, with a sequence of cluster charts. The threshold ηₜ is the minimum percentage of tests which a user has to pass for a confirmation. ηₜ is a value selected from a range of 1.0%≤ηₜ≤97.8% for the preparation of database.

FIG. 17A shows that for a threshold of 50%, there are no unidentifiable users. There is one uniquely identifiable user, and a big cluster which contains the remaining n−1 users. FIG. 17B shows that as ηₜ is increased beyond 59%, unidentifiable users start appearing. FIG. 17C shows that at ηₜ of 67%, one unique identifiable user and two unidentifiable users appear. The number of unidentifiable and uniquely identifiable users rapidly increases beyond a threshold of 70% as shown in FIGS. 17D-17F.

A quantitative analysis for the clusters for δ=3 with an increase in ηₜ is shown in Table 1. The first occurrence of unidentified users took place around ηₜ=59%, and the user clusters between 50.0%≤ηₜ≤95.0% have been analysed. Number of clusters with more than a single user, number of uniquely identifiable users, and number of unidentifiable users are noted for every ηₜ. It is observed that the number of clusters increases with an increase in ηₜ, and the count of both uniquely identifiable and unidentifiable users also increases. Therefore, an optimal threshold value should exist for a given size of users' database, where there would be no unidentifiable users. This value should be chosen by the algorithm as the users' database grows.

A quantitative cluster analysis for δ=3 is presented in Table 1. One of the most important parameters, the cluster connectivity strength, κ is defined as,

$$k = \frac{Number of linkages present in a cluster}{Total number of possible linkages}$$

wherein, κ ranges between 0<κ≤1. Based on the value of κ, a given cluster is classified as either poorly connected (0<κ≤0.33), moderately connected (0.33<κ≤0.66), or strongly connected (0.66<κ≤1.00). Fully connected clusters are those with κ=1.00. The size of the largest cluster in each chart is noted, and the cluster connectivity strength for those clusters are computed. The total possible linkage in this case is equal to ^{m}C₂, wherein m is the number of nodes present in the largest cluster. The connectivity strengths of these large clusters are found to fall in multiple ranges.

TABLE 1
A quantitative cluster analysis for δ = 3.

| Threshold | | | | | |
|---|---|---|---|---|---|
| confidence | Clusters | Number of | | | |
| of confirmation, | with more | uniquely | Size of | Number of | Cluster |
| ηₜ (% | than one | identifiable | largest | unidentifiable | connectivity |
| of users) | user | users | cluster | users | strength, κ |
| 50.00 | 1 | 0 | 94 | 0 | 0.059 |
| 54.09 | 1 | 0 | 94 | 0 | 0.055 |
| 58.18 | 1 | 0 | 93 | 1 | 0.051 |
| 62.27 | 1 | 1 | 92 | 1 | 0.044 |
| 66.36 | 1 | 1 | 91 | 2 | 0.037 |
| 70.45 | 2 | 6 | 81 | 4 | 0.036 |
| 74.55 | 4 | 9 | 66 | 10 | 0.041 |
| 78.64 | 7 | 11 | 27 | 14 | 0.108 |
| 82.73 | 10 | 19 | 14 | 27 | 0.176 |
| 86.82 | 9 | 15 | 9 | 46 | 0.250 |
| 90.91 | 6 | 21 | 3 | 58 | 0.667 |
| 95.00 | 4 | 16 | 2 | 70 | 1.000 |

Table 1 shows that as ηₜ increases from 50%, the connectivity strengths decrease until ηₜ≈74% and increase thereafter with increase in ηₜ, even though the clusters remain moderately connected. Beyond ηₜ≈90%, the largest cluster becomes smaller and strongly connected.

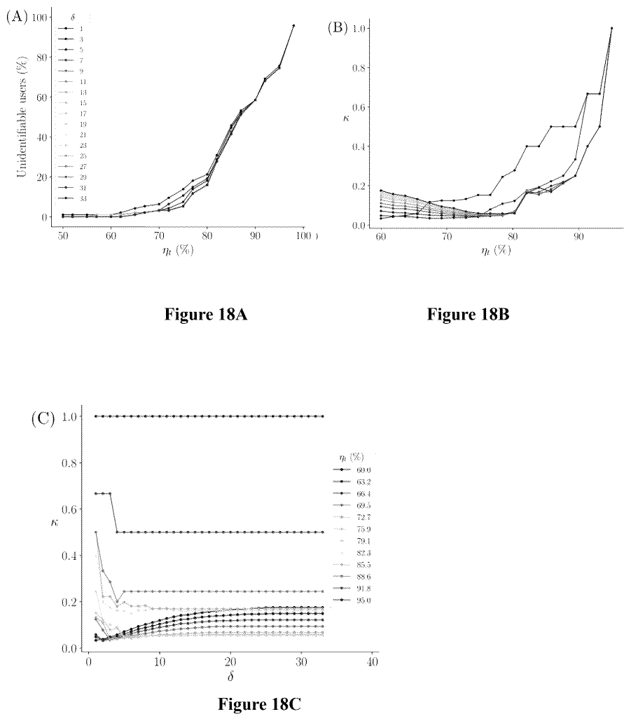

FIG. 18A shows the variation of unidentifiable users (in %) with ηₜ for different values of δ. The behavior of the profile remained unchanged for larger values of δ. The percentage of unidentifiable users increases rapidly only beyond ηₜ≈70%. FIG. 18B shows the variation of the cluster connectivity strength (κ) as a function of ηₜ for different values of δ. For smaller values of δ, κ is observed to remain nearly constant until a critical value of ηₜ, beyond which it increases. This behaviour is slowly transitioned into a point of δ where κ steadily decreases and eventually increases after a certain value of ηₜ. Moreover, for higher values of δ, the curves overlap, and the behaviour remains constant.

FIG. 18C shows the variation of the cluster connectivity strength (κ) as a function of parity parameter (δ) for different values of ηₜ. The larger the value of δ, the greater the dissimilarity between the user pairs. The curves of FIG. 18C provide a good qualitative picture of the effect of δ on κ. The clusters grow to their full size and the linkages remain constant beyond a certain value of δ. As ηₜ increases, this effect becomes more noticeable for smaller δ values. ηₜ=95% indicates the largest cluster wherein users are fully connected for any value of δ.

The novel biometric system works based on the turbulence information present in human exhaled breath. The use of a hot-wire anemometer for data acquisition allowed to build a compact working setup. The real-time computation in combination makes the setup implementable as a biometric authentication system. Based on the user confirmation tests, the machine learning procedure can be used to build a working user confirmation system as it produces good accuracy in confirming users. It achieved a true confirmation rate of nearly 100%, which is because of the ability of random forest models to capture complex decision boundaries between the classes. A multi-model approach is used for the user identification in accordance with various embodiments of the present invention. The outcomes from user identification algorithm, is used to identify two-way connectivity between users, which allows visualization of clusters among the users, which helps in personalized medication. The clustering procedure is proposed as a database-based tool for identifying user clusters, potentially serving as a diagnostic tool when health baseline data is available.

The present invention provides a real-time biometric system which is built solely on the fluid dynamics of exhaled breath wherein said exhaled breath velocity signals that are unique for an individual can be considered as a biomarker and stored in a database for future application. The findings from the clustering analysis performed in accordance with the embodiments of the present invention, could help group users on the basis of the exhaled breath velocity signals. Classification of breathers based on similarity in breathing dynamics can be labeled to personalize the medication prescribed to them.

The biometric system provided by the present invention, may also be used in conjunction with any other type of biometric system such as heart-rate, iris, fingerprint, gait analysis, or the like.

The present invention provides a method for user authentication based on exhaled breath velocity time series signals. The instantaneous velocity time series is measured using a ‘Hot wire anemometer (HWA)’ (2). The exhaled breath velocity time series signals are measured using the HWA (2) when the user exhales through a mouth-piece (1) that is connected to the HWA (2). The velocity time series signals acquired from the collected exhaled breath from a group of users by using a data acquisition system (3) are segmented, filtered, and normalized to extract the features that include an abscissa corresponding to the spectral maxima (β), a width of the spectrum (ω), and a bias or asymmetry parameter of the spectrum (ϵ). Binary random forest classifiers are used for selecting said features. The extracted time series features are utilized to build a model library. This is used as training data and later used for authenticating a user based on this trained data. Data is derived from exhaled breath velocity time series signals to train the random forest models. Data related to breath measurement signals is selected from HWA (2), Laser Doppler Velocimetry (LDV) data, Particle Tracking Velocity (PTV), Particle Imaging Velocimetry (PIV) data, or the like. A particular user is confirmed from among a group of users by comparing the exhaled breath data obtained from the user with the data from the model library and finding specific matching data. The confirmation of the user is based on the random forest models configured to capture complex decision boundaries between classes. The user is identified without prior declaration of user identity by comparing the exhaled breath data of the user with the data from the model library. A multi-model approach for user identification is implemented using a user confirmation block comprising of a hypothesis test based model or machine learning based model.

The method of the present invention is applicable for user authentication using an exhaled breath velocity time series based biometric system, either alone or in combination with other biometric systems such as heart-rate, fingerprint, gait analysis, face, iris, retina, speech or voice, or the like, or in combination with other time series input signals such as body temperature, heart-rate, speech or voice, breathing rate, brain signals, or the like. Also, classification of users done using user identification method can support diagnosis that would aid in personalized medication and treatment.

It is to be understood that “velocity time series” and “time series velocity” have been interchangeably used herein, and are to be interpreted as time series of breath velocity.

It is to be understood, however, that the present invention would not be limited by any means to the techniques described, and any change, variations, and modifications is made without departing from the spirit and scope described in the present invention.

1-16. (canceled)

17. A method for user authentication based on exhaled breath, comprising of a mouth piece (1), a hot wire anemometer (HWA) (2), and a data acquisition system (3),
characterized in that
the method is an exhaled breath velocity time series signals based method, and comprises:
a. collecting exhaled breath from a group of users through the mouth piece (1), the mouthpiece (1) being connected to the HWA (2) for the measurement of exhaled breath velocity time series signals;
b. acquiring velocity time series signals from the collected exhaled breath from the group of users using the data acquisition system (3);
c. segmenting, filtering, and normalizing the acquired velocity time series signals of exhaled breath;
d. extracting a plurality of features from the normalized velocity time series signals;
e. building a model library comprising the features extracted in step d, and generating training data; and
f. authenticating a user based on the training data generated in step e;
wherein, authenticating the user comprises:
confirming the user from the group of users by comparing exhaled breath data obtained from the user with data from the model library for specific matching data; and
identifying the user without prior declaration of user identity by comparing the exhaled breath data of the user with data from the model library.

18. The method as claimed in claim 17, wherein the extracted features include an abscissa corresponding to the spectral maxima (β), a width of the spectrum (ω), and a bias or asymmetry parameter of the spectrum (ϵ).

19. The method as claimed in claim 17, wherein binary random forest classifiers are used for selecting features.

20. The method as claimed in claim 17, wherein data derived from velocity time series signals, either alone or in combination with other signals associated with or unrelated to breath related measurements, is used to train the random forest models.

21. The method as claimed in claim 20, wherein data related to breath measurement signals is selected from HWA (2), Laser Doppler Velocimetry (LDV) data, Particle Tracking Velocity (PTV), Particle Imaging Velocimetry (PIV) data, or the like.

22. The method as claimed in claim 17, wherein the confirmation of the user is based on random forest models configured to capture complex decision boundaries between classes.

23. The method as claimed in claim 22, wherein a multi-model approach for user identification is implemented using a user confirmation block comprising of a hypothesis test based model or machine learning based model.

24. The method as claimed in claim 17, wherein said method is applicable for user authentication using an exhaled breath velocity time series based biometric system individually, or in combination with other biometric systems selected from heart-rate, fingerprint, gait analysis, face, iris, retina, speech or voice, or the like, or in combination with other time series input signals selected from body temperature, heart-rate, speech or voice, breathing rate, brain signals, or the like.

25. The method as claimed in claim 17, comprises classification of users using user identification method, wherein the classification supports diagnosis for personalized medication and treatment.
